(19) United States
(12) Patent Application Publication
(10) Pub. No.: US 2009/0005708 A1
(43) Pub. Date: Jan. 1, 2009

(54) Orthopaedic Implant Load Sensor And Method Of Interpreting The Same

(76) Inventors: Norman A. Johanson, Wynnewood, PA (US); Douglas L. Cerynik, Wynnewood, PA (US)

Correspondence Address: VOLPE AND KOENIG, P.C., UNITED PLAZA, SUITE 1600, 30 SOUTH 17TH STREET, PHILADELPHIA, PA, 19103, US

(21) Appl. No.: 12/164,744

(22) Filed: Jun. 30, 2008

Related U.S. Application Data
(60) Provisional application No. 60/947,201, filed on Jun. 29, 2007.

(51) Int. Cl. A61B 5/103; A61F 2/38

(52) U.S. Cl. 600/587; 623/20.21

(57) ABSTRACT

Joint implant sensors, methods of using the same, and methods of aligning permanent implants are described herein. A device for providing intraoperative in vivo diagnostics of loads having at least one load sensor associated with the implant, and at least one signal processing device operatively coupled with the sensors. The signal processing device is operable to receive the output signal from the sensors and transmit a corresponding signal.

### CROSS REFERENCE TO RELATED APPLICATION(S)

This application claims the benefit of U.S. Provisional Application No. 60/947,201, which was filed on Jun. 29, 2007 and is incorporated by reference herein in its entirety as if fully set forth.

### FIELD OF INVENTION

The present invention relates to diagnostic medical instruments, procedures, trial implant devices and methods for monitoring physiological parameters, and in some embodiments, to arthroplasty and sensors, methods, and implementing software that provide quantitative data for contact between joint surfaces and artificial joint implant devices.

### BACKGROUND

Arthritis, including osteoarthritis (OA), and rheumatoid arthritis (RA), often causes joint damage that leads to severe joint pain and impaired functionality. The procedure of replacing knee joints affected by osteoarthritis and other diseases originated in the early 1960's.

A Total Knee Arthroplasty (TKA) procedure is often performed on a patient suffering from patellofemoral arthritis, or arthritis that is primarily focused around the patella (kneecap) and femur (thigh bone). It is estimated that 478,000 Total Knee Arthroplasty (TKA) operations are performed annually in the United States. While the success rate of this procedure has improved tremendously over the past several decades, revision is still required in a significant number of these cases. About 22,000 of these replacements must be revised each year. Even more revisions are predicted for other joint revision surgery.

The classic approach to determining soft tissue balance during a TKA procedure is the “no thumbs test” which assesses the tracking of the patellofemoral articulation after the implantation of trial components. After putting the joint through a range of motion, the patella visibly lifts off the medial femoral surface and a lateral release is performed. Post-operatively, a tangential patellar view may confirm that the patella is tending to track optimally.

As many as half of all revision TKA procedures are due to complications resulting from patellofemoral resurfacing. Most of these complications are caused by errors in surgical technique, poor prosthetic design, or excessive patellofemoral loads of up to seven or eight times body weight during certain activities such as squatting. In many cases, however, poor knee kinematics and an inadequate understanding of the forces exerted on the prosthetic components play a key role in the wear, mal-alignment, or design flaws associated with these complications.

Patellofemoral complications are a prominent cause of failure in a TKA procedure. Many complications lead to patellar component failure are patellofemoral subluxation (dislocation of the patella to either the medial or lateral side of the knee), which occurs in up to 29 percent of some series, resulting in patellofemoral pain and crepitus, component wear, failure, loosening and/or fracture, malposition of the femoral, tibial or patellar components, poor implant design, patellar fracture, mal-alignment, inadequate patellar resection, avascular necrosis, and revision TKA. Such complications induce many surgeons to avoid patellar resurfacing in patients with osteoarthritis and good remaining articular cartilage. However, several studies indicate increased patellofemoral problems without resurfacing, and secondary resurfacing after primary TKA with a failed non-resurfaced patella, has proven inferior to resurfacing at the time of primary TKA.

Lee et al. describe the association of subluxation and imbalances in mediolateral loading due to soft tissue and bony abnormalities. See Lee, Thay, et al. “The effects of tibial rotation on the patellofemoral joint: Assessment of the changes in in situ strain in the peripatellar retinaculum and the patellofemoral contact pressures and areas.” The Journal of Rehabilitation Research and Development. 38 (2001): 463-469.

Other studies highlight natural knee articulation and describe a gradual medial tilt and lateral shifting of the patella, as well as increasing discrimination in condylar depth and radius, and patellar groove width as deeper flexion is achieved. See Moro-oka, Takaaki, et al. “Patellar Tracking and Patellofemoral Geometry in Deep Knee Flexion.” Clinical Orthopedics and Related Research. 394 (2002): 161-168. Such changes in tracking position and geometry have significant impact on contact area and resultant pressures, and may serve as points of distinction between natural knee and replacement knee kinematics. In the quantification of joint loading, past studies have involved partial cadaveric knees set in mechanical testing equipment and the use of pressure-sensitive Fuji films accurate within only 10%.

Wasielewski has adapted an FDA approved pressure sensor matrix array (Novel® Electronics, Inc., Munich, Germany) for intra-operative measurement of pressure distribution between the medial and lateral tibio-femoral compartments of a posterior stabilized total knee replacement. See Wasielewski, Ray, Daniel Galat, and Richard Komistek. “Correlation of compartment pressure data from an intraoperative sensing device with postoperative fluoroscopic kinematic results in TKA patients.” The Journal of Biomechanics. 38 (2005): 333-339. See also Wasielewski, Ray, Daniel Galat, and Richard Komistek. “An Intraoperative Pressure-Measuring Device Used in Total Knee Arthroplasties and Its Kinematics Correlations.” Clinical Orthopedics and Related Research. 427 (2004): 171-178. Wasielewski showed, in combination with a “balanced gap” technique, a correlation between excessive medial or lateral pressure and the occurrence of lift-off seen in fluoroscopic, kinematics studies performed 6-10 months post-operatively. He concluded that the pressure sensors substantiated intra-operative findings of ligament imbalance, and identified variation of otherwise unrecognizable inequality in pressure distribution. He also concluded that such inequalities may be linked to a long-term impact on prosthetic wear and overall clinical performance.

In contrast to Wasielewski's study, the patellofemoral joint in TKA has not been studied significantly with respect to wear generation, although increased efforts have yielded design modifications with the goal of optimized patellar tracking, pain reduction and functional improvement. As these modifications are based largely on theoretical considerations, present clinical outcome data has not provided significant substantiation.

Previous sensor matrix arrays utilize capacitive, rather than resistive, circuit elements to correlate mechanical deformation with force and/or pressure to quantify patellofemoral loading. These arrays, however, are placed between native bones. Because they are an addition to the joint, the sensor itself may lead to artifact.

A need still exists to improve implant selection, positioning, and design, as well as better understanding of the in vivo forces of the components as they relate to each other, the bone, and the surrounding soft tissue structures. There is also a need to improve knee prosthesis mechanical and wear characteristics, such that the prosthesis may be expected to last a lifetime, and to provide tools with which physicians can perform diagnostics, during surgery, on prosthesis implanted within a patient. There is a need for devices, methods and protocols for joint and bone alignment and tracking for preliminary tests during joint replacement surgery.

### SUMMARY

In one aspect, the present invention relates to a sensor device. The sensor device includes a base plate having a base plate bottom surface adapted to contact a joint surface, and a base plate top surface. The sensor device also includes a conformable sensor including a sensor matrix that includes individual sensor elements, and a conformable mat. The conformable mat supports the sensor matrix. The sensor device also includes an implant having an implant bottom surface and an implant top surface. The conformable sensor is operably connected to the implant and is associated with either the base plate top surface, the implant bottom surface, the implant top surface, or the base plate top surface and the implant bottom surface. Also, the base plate top surface faces and is operatively connected to the implant bottom surface.

In a second aspect, the present invention relates to a sensor device comprising a base plate having a base plate bottom surface adapted to contact a joint surface and a base plate top surface. The sensor device also includes a conformable sensor matrix that includes individual sensor elements arranged in a conformable mat. The sensor device also includes an implant having an implant bottom surface and an implant top surface. The conformable sensor is operably connected to the implant and associated with either the base plate top surface, the implant bottom surface, the implant top surface, or the base plate top surface and the implant bottom surface. The base plate top surface faces and is operatively connected to the implant bottom surface.

In a third aspect, the present invention relates to a method of using a sensor device to measure joint characteristics during joint replacement or joint implant revision. The method includes providing a sensor device including a base plate having a base plate bottom surface adapted to contact a joint surface, and a base plate top surface. The provided sensor device also includes a conformable sensor that has a sensor matrix that includes individual sensor elements and a conformable mat. The conformable mat supports the sensor matrix. The provided sensor device also includes an implant having an implant bottom surface and an implant top surface. The conformable sensor is operably connected to the sensor implant and associated with either the base plate top surface, the implant bottom surface, the implant top surface, or the base plate top surface and the implant bottom surface. The base plate top surface faces and is operatively connected to the implant bottom surface. In this aspect, the method further includes making an incision in a patient to expose the joint and removing one of the group consisting of bone and pre-existing implants. The method also includes inserting the sensor device and remaining joint implants required for the joint replacement or joint implant revision into the joint, moving the joint through a partial or full range of motion, collecting joint data through the sensor matrix, making necessary adjustments based on the joint data, and repeating testing, data collection and adjustments as necessary until the joint, the sensor implant, and the remaining joint implants are in a desirable position. The method also includes removing the sensor device, inserting a final implant in place of the sensor device, and closing the incision.

In a fourth aspect, the invention relates to a sensor device comprising a base plate having a base plate bottom surface adapted to contact a joint surface, and a base plate top surface. The sensor device also includes a conformable sensor including a sensor matrix that includes individual sensor elements, and a conformable mat. The conformable mat supports the sensor matrix. The sensor device also includes an implant having an implant bottom surface and an implant top surface. The conformable sensor is operably connected to the implant and is associated with either the base plate top surface, the implant bottom surface, the implant top surface, or the base plate top surface and the implant bottom surface. The base plate top surface faces and is operatively connected to the implant bottom surface. The sensor device further includes a transceiver and antenna to wirelessly transmit data to a data processor or wirelessly receive communications.

In a fifth aspect, the present invention relates to a joint replacement implant collection. The collection includes a sensor device shaped in the form of a final implant that will be associated with one bone within a joint. The sensor device includes a base plate having a base plate bottom surface adapted to contact a joint surface and a base plate top surface. The sensor device also includes a conformable sensor including a sensor matrix that includes individual sensor elements and a conformable mat. The conformable mat supports the sensor matrix. The sensor device also includes an implant having an implant bottom surface and an implant top surface. The conformable sensor is operably connected to the implant and is associated with either the base plate top surface, the implant bottom surface, the implant top surface, or the base plate top surface and the implant bottom surface. The base plate top surface faces and is operatively connected to the implant bottom surface. In this aspect, the collection also includes remaining implants in the form of implants associated with the remaining bones in the joint.

In a sixth aspect, the present invention relates to a joint replacement implant collection. The collection includes a sensor device shaped in the form of a final implant that will be associated with one bone within a joint, the sensor device including a base plate having a base plate bottom surface adapted to contact a joint surface and a base plate top surface. The sensor device also includes a conformable sensor including a sensor matrix that includes individual sensor elements arranged in a conformable mat. The sensor device also includes an implant having an implant bottom surface and an implant top surface. The conformable sensor is operably connected to the implant and is associated with either the base plate top surface, the implant bottom surface, the implant top surface, or the base plate top surface and the implant bottom surface. The base plate top surface faces and is operatively connected to the implant bottom surface. In this aspect, the collection also includes remaining implants in the form of implants associated with the remaining bones in the joint.

### BRIEF DESCRIPTION OF THE DRAWING(S)

The following detailed description of the preferred embodiments of the present invention will be better understood when read in conjunction with the appended drawings. For the purpose of illustrating the invention, there are shown in the drawings embodiments which are presently preferred. It is understood, however, that the invention is not limited to the precise arrangements and instrumentalities shown.

FIG. 1 illustrates a perspective view of a healthy human knee in an unflexed position.

FIG. 2 illustrates a front view of a healthy human knee in a flexed position.

FIG. 3 illustrates a perspective view of a human knee with osteoarthritis.

FIG. 4a illustrates a side view of a resurfaced human knee undergoing a Total Knee Arthroplasty procedure when a femoral implant is attached to the femur.

FIG. 4b illustrates the resurfaced human knee of FIG. 4a, with a tibial implant attached to the tibia.

FIG. 4c illustrates the resurfaced human knee of FIG. 4b, with a patellar implant attached to the patella.

FIG. 5 illustrates a perspective view of a resurfaced human knee with a replacement implant.

FIG. 6 illustrates a top view of a sensor.

FIG. 7 illustrates an exploded view of the first embodiment of the sensor device, with the sensor disposed between bottom plate and the implant surface.

FIG. 8 illustrates an exploded view of the second embodiment of the sensor device, with the sensor embedded within the implant surface.

FIG. 9 illustrates an exploded view of the third embodiment of the sensor device, with the sensor disposed on top of the implant surface.

FIGS. 10 and 11 illustrate a display of the pressure and/or force data after being collected by the sensor and processed by the software.

FIG. 12 illustrates a method of using the sensor device of the present invention during a surgical procedure to replace a joint.

FIG. 13 illustrates a method of using the sensor device of the present invention during a surgical procedure to revise a joint replacement.

### DETAILED DESCRIPTION OF THE PREFERRED EMBODIMENT(S)

Certain terminology is used in the following description for convenience only and is not limiting. The words “right,” “left,” “top,” and “bottom” designate directions in the drawings to which reference is made.

The words “a,” and “one,” as used in the claims and in the corresponding portions of the specification, are defined as including one or more of the referenced item unless specifically stated otherwise. This terminology includes the words above specifically mentioned, derivatives thereof, and words of similar import.

The term “joint surface” refers to bone or cartilage surfaces within a joint or such surfaces near a joint.

As used herein, the terms “trial component” and “sensor device” are used interchangeably.

As used herein, loading forces means the forces placed upon a joint.

As used herein, joint data means data regarding the impact of loading a joint and can take the form of force, stress, or pressure measurements. Joint data may be collected by a sensor device and pertain to a surface of interest on the sensor device, within the sensor device, or to indirect forces applied to the sensor device.

As used herein, “patient” refers to any human or non-human animal subject.

A preferred embodiment of the invention includes a sensor device for intraoperative use during orthopedic implant surgery. The sensor device allows at least external monitoring of the i) force between an orthopedic implant or other medical devices and the patient, ii) force or pressure between a joint trial component and the underlying bone, iii) forces internal to a medical device, iv) force or pressure between a trial component and other orthopedic components, v) forces or pressures of surrounding soft tissue structures on the trial component. Examples of medical devices with which this invention can be used consist of, but are not limited to the following: a) the tibial, femoral, or patellar components used in total knee replacement, b) the femoral or acetabular components used in total hip implants, c) the scapular or humeral components in shoulder replacement, d) the tibia and talus in ankle replacement, and e) devices implanted between the vertebral bodies in lumbar or cervical spine disk replacements. Observing the forces in the joint will allow surgeons to better understand the kinematics of the joint, the effects of load magnitude, or load imbalance. Based on these understandings, a surgeon can make adjustments regarding component selection, component position, or soft tissue procedures that need to be performed, intraoperatively. Data from sensing elements will provide accurate boundary conditions for mathematical models that affect the joint. Sensing data will help joint replacement implant manufacturers better understand the real-time operative forces or pressures seen by the joint and more accurately produce implant components.

In a preferred embodiment, a sensor element or elements are arranged in a mat. In another embodiment, the sensor element or elements are supported by a conformable mat. The mat may conform to the underside or top side of an implant surface. Alternatively, the mat and sensor may be integrally formed or embedded within the implant surface. In a preferred embodiment, individual sensor elements are provided within a sensor matrix. The conformable mat can support the sensor matrix by providing a surface for the matrix to rest on or adhere to. The conformable mat can also support the sensor matrix by providing a material in which at least a portion of the matrix is embedded.

A sensor can be powered by electromagnetic induction, radio frequency (RF) induction or batteries. The sensor can use RF technology or other means to remotely or wirelessly transmit data. A non-remote version may exist in which the device is powered externally and transmits data via wires.

One embodiment of the device provides in vivo diagnostics of loads in orthopedic implants. The device has at least one load sensor associated with the implant for generating an output signal in response to and indicative of normal and transverse loads being applied to the implant. At least one signal processing device is operatively coupled with at least one load sensor and receives output signals from at least one load sensor. The signal processing device also transmits a signal corresponding to the output signal.

In an embodiment, the device is used to quantify the loading condition of a joint intraoperatively by sensing, measuring and depicting the forces and pressures existing between the human body or limb and prosthetic implants. Preferably, the present invention is used to measure the patellofemoral bearing surface pressures and forces during a TKA procedure to provide data to a surgeon during surgery and permit the surgeon to make ongoing adjustments to the joint surface and/or implant prosthesis during surgery.

Patellofemoral joint balancing during a TKA surgical procedure can be maximized using objective joint load measurements produced by ligamentous constraints and muscle forces. To achieve this result, the embodiments described herein provide objective criteria for evaluating surgical techniques (e.g. standard vs. “mini” approaches, effectiveness of performing a lateral release), objective criteria for evaluating existing prosthetic designs, and design criteria for developing new implants.

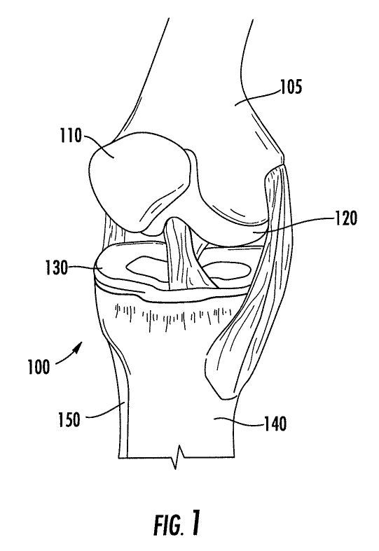

Referring to FIG. 1, which illustrates a healthy human knee joint 100 in an unflexed state, three major bones make up a knee joint. These bones are the femur 105 (thigh bone), the tibia 140 (shin bone), the fibula 150, and the patella 110 (knee cap). The patella 110 faces the front surface 120 of the femur 105. The meniscus 130 is an area of cartilage that separates the femur 100 and the tibia 140. The meniscus 130 also absorbs and disperses the pressure imposed by a person's weight so that the femur and tibia do not rub together.

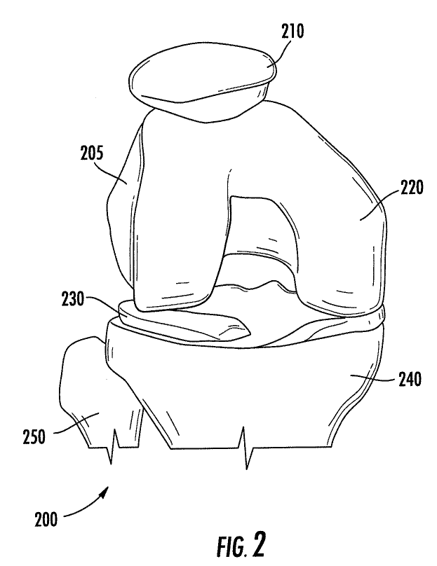

FIG. 2 shows a front view of a healthy human knee joint 200 in a flexed position, including the femur 205, the front surface 220 of the femur, the tibia 240, the fibula 250, the patella 210, and the meniscus 230. As the knee joint moves from a straightened position, unflexed state to a flexed position, the front surface 220 of the femur and the patella 210 both rotate and face upward.

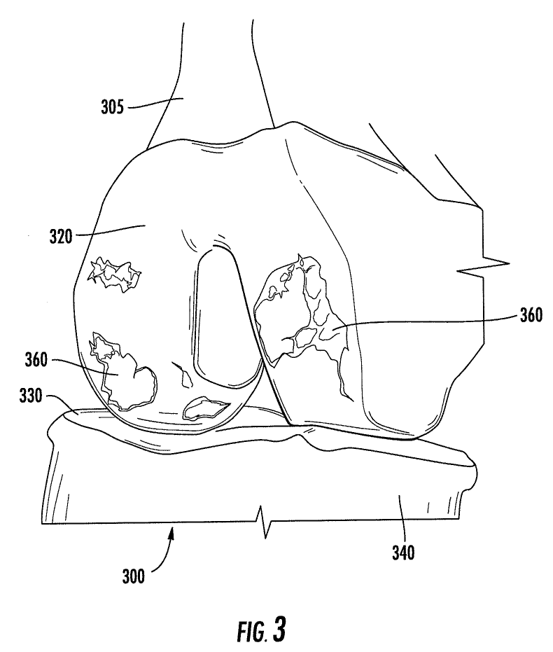

Referring to FIG. 3, knee joints that succumb to arthritis, such as osteoarthritis (OA) and rheumatoid arthritis (RA), often develop bone spurs or areas of worn, exposed bone 360. When knee joints 300 develop arthritis, the meniscus 330 wears away, allowing the femur 305 and tibia 340 to rub together. The friction between the femur 305 and tibia 340 rubbing together may form areas of wear 360 on the end 320 of the femur 305 and tibia 340. Knee joint replacement procedures are often performed to replace a knee with this type of wear, regardless of its source.

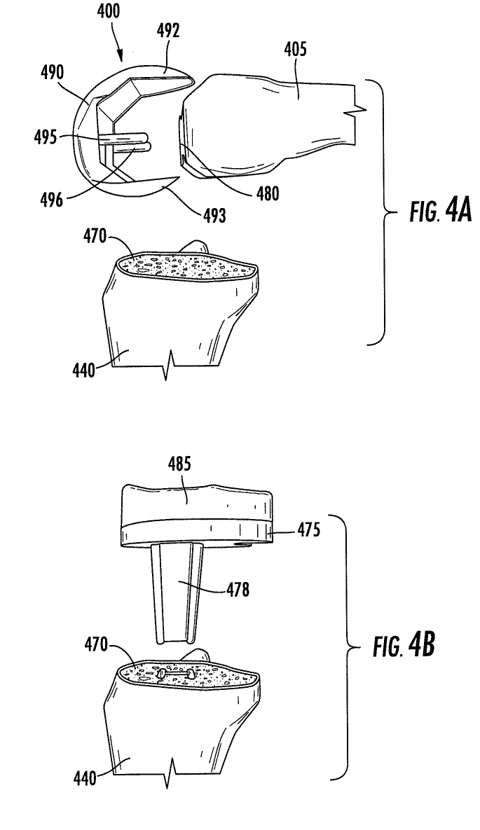

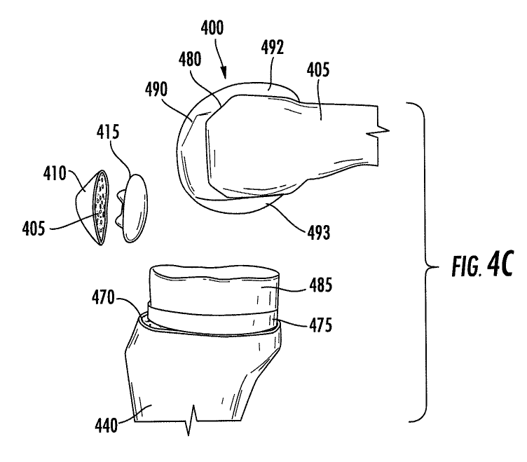

A TKA procedure involves removing the worn, exposed bone areas 360 on the femur 300 and/or tibia 340, reshaping the remaining bones, and replacing these damaged bone areas with new, durable artificial implant devices prosthesis. The femur and tibia must be reshaped to ensure they fit properly with the new knee implant prosthesis. An embodiment of a TKA procedure is described with reference to FIGS. 4-6, below.

Referring to FIGS. 4a, 4b, and 4c, a knee joint replacement procedure is generally performed as follows. When the leg is in an extended position and the knee is in an unflexed state, an elongate incision is made in the front of the knee. The tissue surrounding the incision is then cauterized and folded out of the way to expose the knee joint 400. The leg is then bent to the proper angle and the knee is elevated. At this point, the knee joint 400 bones are exposed and prepared for resurfacing. The meniscus and any bone spurs are then removed.

Still referring to FIGS. 4a, 4b, and 4c, the femur 405, tibia 440, and patella 410 are then reshaped and prepared to receive new knee implant prosthesis. A hole is drilled in the femur 405 to set up alignment devices. A desired portion of the femur 400 is then cut off and reshaped, as shown in FIG. 4a. A similar alignment and reshaping process is done to the tibia 440, as shown in FIG. 4b. The patella 410 surface is also cut and reshaped to prepare the area for receiving an implant prosthesis, as shown in FIG. 4c.

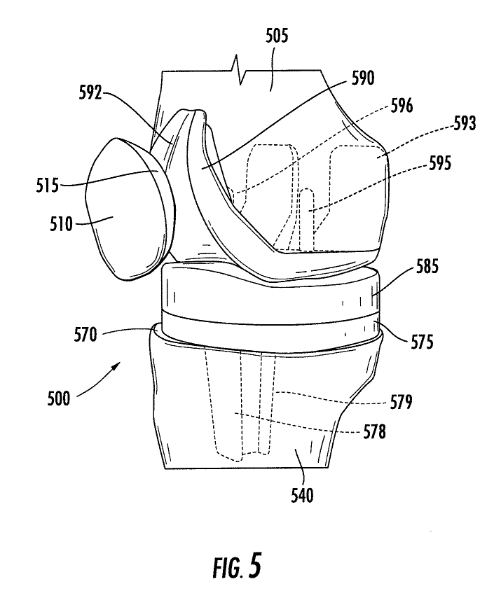

Referring to FIG. 4a, desired portions of the femur 405 and tibia 440 are cut off to form respective flat implant receiving surfaces 480, 470. A femoral implant 490 is then attached to the femur at the femur's flat implant receiving surface 480. The femoral implant 490 includes pins 495, 496 and teeth 492, 493 to secure the femoral implant 490 onto the femur 400 at the femur's receiving surface 480. Referring to FIG. 4b, a metal tray implant 475 with a plastic spacer 485 is attached to the tibia 440 at the tibia's receiving surface 470. The metal tray implant 475 has teeth 478 (see also FIG. 5, teeth 578, 579) that secure the metal tray implant 475 into the tibia 440 at the tibia's receiving surface 470.

Referring to FIG. 4c, patellar implant 415 is attached to the patella 410 at the patella's reshaped surface 405. FIG. 4c shows an exploded view of the femur, tibia, and patella with their respective attached implant prosthesis, before being re-attached together.

FIG. 5 shows the knee joint 500 with the complete implant knee prosthesis, where the leg is extended and the knee joint is in an unflexed state. The femoral implant 590 faces and abuts the plastic spacer 585 on the metal tray implant 575. The femoral implant's teeth 595, 596 extend upward into the femur and the metal tray implant's teeth 578, 579, extend downward into the tibia. The plastic spacer 585 separates the femoral implant 590 and the metal tray implant 575, which prevents the femur 505 and tibia 540 from rubbing together and causing wear spots due to friction. The plastic spacer 585 also absorbs and disperses the pressure imposed by a person's weight so that the femur and tibia do not rub together.

In a preferred embodiment, after inserting the components, a surgeon tests the knee joint's range of motion intraoperatively by elevating and lowering the knee, bending and extending the leg, and ensuring there are no gaps between the femoral and tibial implants. Testing the joint's range of motion ensures the implants have not been mal-aligned, which could lead to adverse complications post-surgery.

In a preferred embodiment, after testing the implant prosthesis, the implant components are removed and prepared for permanent insertion. Cement is applied to the components, which are then re-inserted and placed into their permanent positions. The cement is allowed to harden, and range of motion tests are then performed again before the incision is closed and surgery is complete.

In a preferred embodiment, a sensor device is used in conjunction with an artificial joint implant to provide quantitative data for contact between bones and an implant during orthopedic implant surgery. The sensor may also indirectly read the pressures, strains, or forces that the soft tissue places on the implant. A surgeon performing a joint replacement procedure can use this data to make necessary adjustments to the implants, bones, or associated tissue while performing the procedure, and thus reduce the risk of post operative complications. Intraoperative assessment of knee alignment or stability can include range of motion tests varus/valgus rotation, varus/valgus stress, and joint distraction, in conjunction with the sensor device.

Implants or sensor devices can be made in whole or in part with a material that is forgiving but resistant to wear. The material can be polyethylene, and/or highly-crosslinked polyethylene. In a further embodiment, the implants or sensor devices can be made with a polyethylene and or highly crosslinked polyethylene top portion (e.g., a dome or surface) with a tantalum backing. The skilled artisan will recognize that the choice of materials can be adapted to the properties desired in the implant or sensor device. Preferred embodiments described herein relate to patello-femoral implants. However, other embodiments are envisioned regarding other joint implants.

In an embodiment, the sensor device obtains pressure distribution measurements between soft and curved bone surfaces. The sensor device includes an implant surface and a conformable sensor. In a further embodiment, the conformable sensor includes a data transmission device. The sensor device can include a base plate associated with the implant surface. Present embodiments are also directed to a method of using the test joint implant during a joint replacement procedure or a revision procedure.

In a preferred embodiment, the conformable sensor has elasticity. The elasticity of the conformable sensor permits deformation and conformability to 3-dimensional surfaces. In a preferred embodiment, the conformable sensor also includes individual capacitive transducers arranged in a matrix configuration. The transducers contain high-tech elastomers. Different sizes, configurations, and pressure ranges of the elastic sensors may be used.

In a further embodiment, the sensor device includes analyzer technology that allows individual calibration curves for each sensor and individual dynamic amplification control and crosstalk suppression. In this embodiment, accurate and reproducible pressure values can be reported. The analyzer can be used with a computer via an operable connection such as a USB interface, and wire or wireless communication. Notebook computers or even a pocket PC or other wireless devices can be used for mobile tests. Analyzers ranging from small portable 16×16 channel units to large 112×112 channel units with a wide range of options, such as master-slave synchronization of several systems, dynamic amplification control, synchronization of video systems and analog inputs for accelerometers, may also be used.

In still further embodiments, analyzers and sensors are used in conjunction with a software application on a laptop, desktop, or pocket pc. The software application includes methods for fast force and pressure data collection, analysis, and display. The software, sensors, and analyzers may be used in conjunction with one another to display real-time force and pressure pictures and graphs. Alternatively, the data may be stored on a network or in a database, for example, in an SQL configurable database. A user may customize and design the parameter configuration to meet the user's specific needs. The software application, when used with prosthesis, may be designed to measure and display the pressures and the forces at the limb-socket interface.

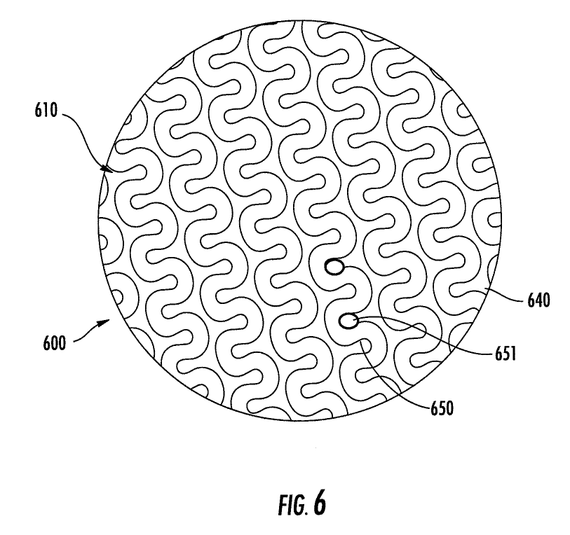

Referring to FIG. 7, in the embodiment illustrated, the sensor device 700 includes a conformable sensor 710 that can detect loads on a curved surface. The conformable sensor 710 includes a sensor matrix 750 with the individual sensor elements 751. The conformable sensor 710 includes a matrix 750 with individual sensor elements 751, and the matrix 750 is associated with a conformable mat 740 and a base plate 720. The conformable sensor 710 is positioned to measure force on an implant surface.

In a preferred embodiment, the conformable sensor 710 includes a force sensor, such as a resistive sensor (for example, Tekscan ISCAN® 5051 sensor), a capacitive sensor (for example, Novel AJP Sensor), a piezoelectric sensor, a force transducer, a strain gauge, a microelectromechanical contact stress sensor. Other sensor types known to one of ordinary skill in the art are also contemplated as alternative embodiments.

In an embodiment, the sensor device includes a sensor matrix comprising force sensors for measuring the distribution of compressive forces over an area within a joint, such as the force sensor described in U.S. Pat. No. 4,862,743 to Seitz. Compressive forces act substantially vertically with respect to a deformable measuring surface. In a preferred embodiment, a matrix arrangement of force sensors forms a capacitance at crossings of substantially perpendicular conductor paths. The conductor paths are fixed on the opposed surfaces of an elastically deformable area-type dielectric and adapted to be connected by conductive elements to evaluator electronics. The conductor paths are printed on plastic substrate films. Such a force sensor includes a plurality of force detectors that include a capacitor. Each capacitor is formed by capacitor elements with a first group of capacitor elements arranged on one surface of an elastically deformable area-type dielectric, and a second group of capacitor elements arranged on a second surface thereof. The capacitors thus are formed at the points of intersection of the first and second capacitor elements. In this manner, a matrix arrangement of force detector means is obtained. The groups of capacitor elements are operatively connected, e.g. by leads, to electronic equipment for evaluation. Together with the leads, the capacitor elements are printed on substrate sheeting or films made of plastics. Simple conductor paths are useful when printed on a plastic substrate film.

The sensor device of a preferred embodiment includes a sensor matrix that includes individual capacitive sensor elements. A capacitive sensor includes a grid of conductive strips fixed, e.g. with glue, on an elastic dielectric material. Each intersection of two active strips results in a capacitor. Under external load, the dielectric thickness decreases, causing a change of the capacitance according to the equation:

$$C = {ɛɛ}_{0} \cdot \frac{A}{d} ( \begin{matrix}where ɛ , {ɛ}_{0} = dielectric constants ; \\ A = plate area ; and d = plate distance\end{matrix} )$$

The changes in capacitance are measured and subsequently transmitted to a processing device for storage or real-time display.

In another embodiment, the sensor device includes a sensor matrix that includes individual resistive sensor elements. A resistive sensor includes two Mylar sheets that have electrically conductive electrodes deposited in varying patterns. Before assembly, a semiconductive coating (ink) is applied as an intermediate layer between the electrical contacts (rows and columns). This ink provides a change of the electrical resistance at each of the intersecting points when pressure is applied. When the two Mylar sheets are placed on top of each other, a grid pattern is formed, creating a sensing location at each intersection. By measuring the changes in current flow at each intersection, an applied force distribution pattern can be measured.

The technologies used to connect the resistor or capacitive sensor elements (sensels) to the signal-conditioning electronics are based on the same principle: a sensor matrix is operatively connected to a multiplexer, which allows reading of the array of parallel sensor elements in a serial manner and displays two-dimensional pressure distribution in real time.

In the preferred embodiments, the characteristics of resistive and capacitive sensors used range between 0.1 mm-1 mm (thickness), 28 mm×43 mm to 56 mm×56 mm (overallis size), 16 sensels/cm²-62 sensels/cm² (resolution), and 2.5 MPa-17.1 MPa (maximum pressure).

Both resistive and capacitive sensors may have different mechanical and shape characteristics due to the materials from which they are manufactured. For example, Mylar is used in the ISCAN® resistive sensor and foam rubber is used in the capacitive AJP sensor. The variation in materials used to make the different sensors accounts for the different sensors' resulting physical properties. The capacitive AJF sensor may be conformed to a surface. The ISCAN® resistive sensor is nearly 10 times thinner than the capacitive AJP sensor. Sensors manufactured by any of these designs are contemplated as embodiments of the present invention.

Either a resistive or a capacitive sensor may be used in the present embodiments. A capacitive sensor has a smaller force detection error margin than a resistive error. A capacitive sensor also has greater repeatability and homogeneity qualities than a resistive sensor. However, the capacitive sensor has greater errors in contact area measurement than the resistive sensor. Also, the resistive sensor tends to crinkle when conformed to a spherical surface. The magnitude of the expected loading, the relative importance of accuracy, repeatability, and conformability to complex geometric features of the given application all affect which sensor is more desirable for a given application. Accuracy, repeatability, and conformability are attained at the cost of resolution and potentially biased area and peak pressure measurements using a thicker sensor, and improved area and pressure distribution measurements at the cost of accuracy and repeatability when using a thinner sensor. Either sensor is suitable measuring forces in a joint.

Alternatively, the sensor device of a preferred embodiment includes a force transducer, such as the one described in U.S. Pat. No. 5,470,354 to Hershberger. A force transducer, when positioned within a joint, may be used to collect data regarding the location and magnitude of the sum of forces generated in the joint when the joint is moved through its range of motion. It measures and pinpoints the loads applied in the joint during rotation and testing. A force transducer positioned within a joint may be operatively linked to a computer terminal. When bearing elements rest on a force sensor, the specific contact areas of a joint or bone component and the bearing element are summed and transferred so that the corresponding area of the force transducer are displayed as a point or line on the data terminal. When the joint is moved through its range of motion, the magnitude and location of the sum of the forces generated in the joint are transferred to the sensor by the bearing elements. These forces in turn are displayed on the data terminal. A force transducer may be thin (on the order of 0.010-0.020 inches in thickness).

Alternatively, the sensor device of another embodiment includes a piezoelectric sensor, such as the sensor described in U.S. Pat. No. 7,097,662 to Evans. A piezoelectric sensor can also be displaced laterally on a transducer to provide independent force data for various locations within a joint.

Alternatively, the sensor device of another embodiment includes a strain gauge for measuring force components within a joint. The strain gauge described in U.S. Pat. No. 5,425,775 to Kovacevic et al. may be included in this embodiment. When force acting on a sensor cover causes a diaphragm-like deflection on an object, a strain gauge detects the force. The strain gauge measures and converts the amount of deflection into electrical signals. The strain gauge is typically connected to wires, which connect the sensor and an apparatus that is capable of calculating the forces on the sensor cover from the electrical signals sent by the strain gages. Strain gages connected in Wheatstone bridges provide signals indicating the magnitude of the force in the direction of each axis. Excitation and readout circuitry is used to provide information to either a display or to a recorder, as desired. Alternatively, optical sensors can be used in place of strain gauges for sensing deflection of a disc and flexure of a support. Wireless communication is also contemplated and, if desired, a radio transmitter can be built into the sensor with a suitable power supply for wireless transmission of force data.

Alternatively, the sensor device of the present invention may include a microelectromechanical systems contact stress sensor, as described in U.S. Pat. No. 7,311,009 to Kotovsky. A microelectromechanical systems contact stress sensor includes a silicon beam with an embedded electric circuit that contains piezoresistor material. This material senses changes in resistance. As the object in which the sensor is placed bends, the silicon beam also bends. The piezoresistor material's resistance also changes proportional to the silicon element's bending. This change in resistance, which is proportional to the change in bending that arises from an applied load, is quantitative load data that may be transmitted to a data processor for further processing. Further processing can include providing the data to a computer navigation system.

Referring to FIG. 6, an embodiment of the sensor device 600 is illustrated. The sensor device 600 includes a conformable sensor 610 and a base plate 720 (not shown). The conformable sensor 610 includes a sensor matrix 650 supported on or in a conformable low friction sensor mat 640. The sensor matrix 650 includes individual sensor elements 651. Connections for reporting joint loading data can be embedded within the sensor mat 640. When in place, the sensor may be operatively connected to a data processor to transmit joint loading data to the processor for subsequent storage, processing, and display.

Referring to FIG. 7, an embodiment of the sensor device 700 is illustrated. The sensor device 700 includes a conformable sensor 710, a dome shaped implant 730, and a base plate 720. The conformable sensor 710 includes a sensor matrix 750 supported on or in a conformable low friction sensor mat 740. The sensor matrix 750 includes individual sensor elements 751. In the embodiment, the sensor mat 740 is placed on a bottom base plate 720, between the base plate 720 and of the patellar implant 730. Connections for reporting joint loading data can be embedded within the sensor mat 740. When in place, the sensor may be connected to a data processor to transmit joint loading data to a processor for subsequent storage, processing, and display. Also depicted are a base plate bottom surface 721, a base plate top surface 722, an implant bottom surface 731, and an implant top surface 732.

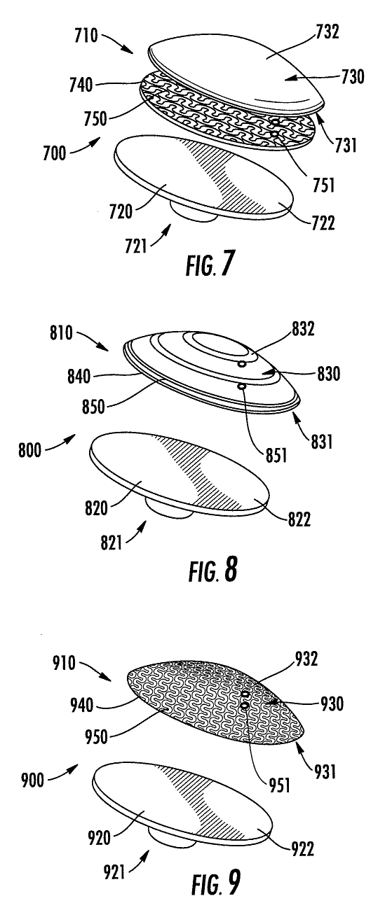

Referring to FIG. 8, another embodiment of the sensor device 800 is illustrated. The sensor device 800 includes a conformable sensor 810, a dome shaped implant 830, and a base plate 820. The sensor 810 includes a sensor matrix 850 supported on or in a conformable low friction sensor mat 840. The sensor matrix 850 includes individual sensor elements 851. In the embodiment illustrated, the conformable sensor 810 is associated with the dome-shaped implant 830 by being embedded within the dome-shaped implant 830. Alternatively, the conformable sensor 810 includes a matrix 850 with its individual sensor elements 851 associated with the implants 830 but with no conformable mat. Connections for reporting joint loading data are embedded within the sensor mat 840. Also depicted are a base plate bottom surface 821, a base plate top surface 822, an implant bottom surface 831, and an implant top surface 832.

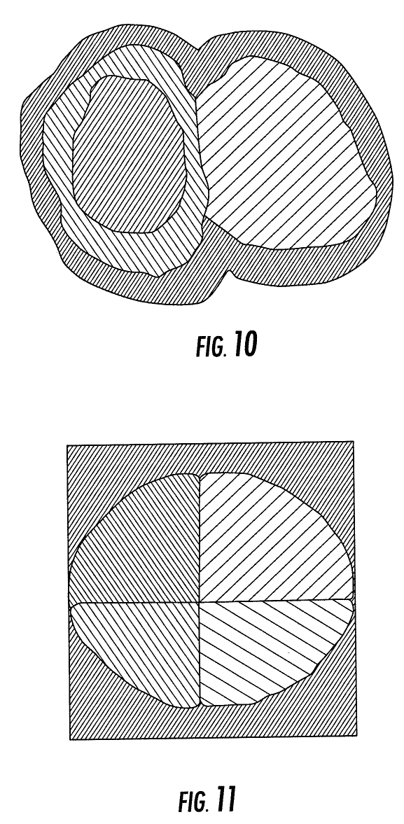

Referring to FIG. 9, another embodiment of the sensor device 900 is illustrated. The sensor device 900 includes a conformable sensor 910, a dome shaped implant 930, and a base plate 920. The conformable sensor 910 includes a sensor matrix 950 supported on a conformable low friction sensor mat 940. The sensor matrix 950 includes individual sensor elements 951. In the embodiment, the conformable sensor 910 is associated with the dome-shaped implant 930 by being placed on the implant's top surface, preferably directly on the implant surface. Alternatively, in another embodiment, the conformable sensor 910 includes a matrix 950 with its individual sensor elements 951 associated with the implant but with no conformable mat. Connections for reporting joint loading data can be embedded within the sensor mat 940. When in place, the conformable sensor 910 may be connected to a data processor to transmit joint loading data to a processor for subsequent storage, processing, and display. Also depicted are a base plate bottom surface 921, a base plate top surface 922, an implant bottom surface 931, and an implant top surface 932.

As described above, the conformable sensor may be positioned between the base plate and implant or above the implant. In still further embodiments, the conformable sensor can be positioned anywhere, including the bottom of the base plate or on or within the sensor device such that the sensor matrix or individual sensor elements are able to sense loading forces on the position of interest on the sensor device. The position of interest may be a surface of the sensor device or within the sensor device. In the different possible positions, the conformable sensor can be associated with one surface or with more than one surface of the sensor device by being placed in contact with the surface, placed between two surfaces, wedged in place, reversibly adhered, irreversibly adhered, at least partially embedded, fully embedded, or being integral with the surface. In some embodiments, as illustrated in FIG. 7, 8, or 9, there can be a base plate bottom surface, a base plate top surface, an implant bottom surface, and an implant top surface. The conformable sensor can be associated with any one of the surfaces or a combination of these surfaces.

In the embodiments illustrated in FIGS. 7, 8, and 9, the conformable sensor is positioned either between the implant and base plate, on top of the implant, or at a position in between. In these embodiments, even when the conformable sensor is disposed between the implant and base plate, the implant and base plate are operably connected. As used herein, the implant and base plate are operably connected when the two parts can by held together to form the implant, conformable sensor, base plate structure. The manner in which the components are held together is not limited but may include the following. The operable connection, in some embodiments, could be through fasteners running from either the implant or the base plate and to the other. The fasteners may be disposed through or around the conformable sensor. In other embodiments, the operably connected implant and base plate are held together with reversible or irreversible adhesives.

In an embodiment, the entire sensor, including the base plate, conformable sensor, and implant are integrally formed as one piece. In another embodiment, a subset of parts of the sensor are integral with one another.

In still further embodiments, the conformable sensor is operably connected with the implant. As used herein, a conformable sensor is operably connected with an implant when the individual sensors or sensor matrix can sense load forces on the implant.

In further embodiments, the sensor device is connected to a data processor to transmit joint loading data to a processor for subsequent storage, processing, and display. The sensor device may be powered externally or internally, through electric wires and cables, electromagnetic induction, radio frequency (RF) induction, or batteries, or any other suitable powering means known in the art. The sensor device may also use wires, or alternatively, RF technology or other connections known in the art to remotely transmit data. Also, the data processor may, but is not limited to, a data processing device, such as a computer, a laptop computer, a remote or hand-held wireless data processing device, a cellular communications device, and a cellular telephone. In an alternative embodiment, the sensor may be connected through, for example, telemetry. As described, the sensor device may be wirelessly connected to a processing device in some embodiments. In such an embodiment, the sensor device and the data processor can be equipped with transceivers and/or antenna along with the associated hardware or software necessary to implement wireless communication, as known in the art. Associated hardware or software can include a microprocessor and RAM. In still further embodiments, the wireless standard implemented may include bluetooth, Global System for Mobile communications (GSM), Enhanced Data rates for GSM Evolution (EDGE), General Packet Radio Service (GPRS), cdma2000, wideband CDMA (W-CDMA), long term evolution (LTE), 802.11x, Wi-Max, or mobile Wi-MAX.

In another embodiment of the invention, a method of performing patellafemoral alignment using a sensor device is achieved. In this method, a sensor device can be placed into a joint during surgery. The sensor device occupies the position intended for a final implant. And remaining implants associated with the entire joint implant are also placed. For example, the sensor device can be a trial component patellar implant and the remaining implants could then include a femoral implant 490, a metal track implant 495, and a plastic spacer 485. After placement, joint loading data is collected, and adjustments are made to the bones, other tissues, or implants based on the data obtained by the sensor.

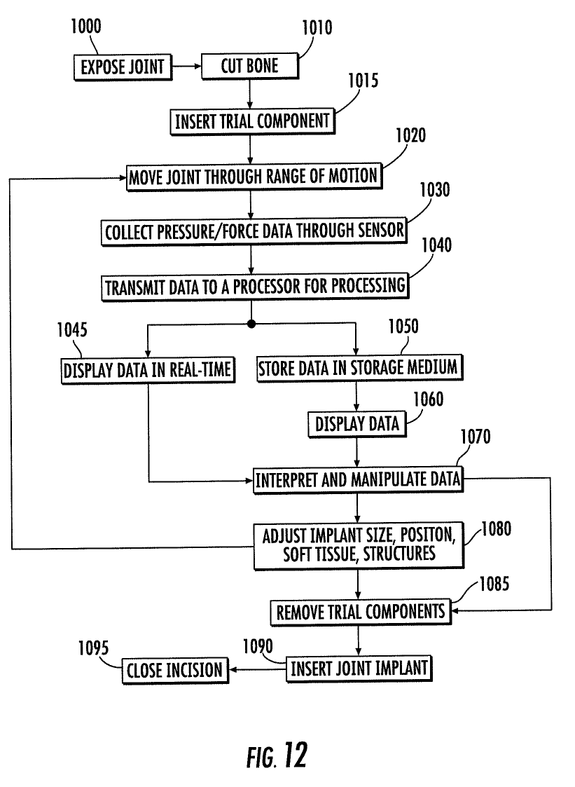

Software may also be used to produce contact area versus time graphs for real-time kinematic observations. Referring to an embodiment illustrated in FIGS. 10 and 11, stress areas may be color coded by type of color and/or shade of color. For example, intense red could indicate high stress, pink for moderately high stress, yellow for medium stress, and blue for low stress. Data may be summed for a selected area of a test implant. As illustrated, the selected area can be each quadrant and the data may be displayed in the colors over each quadrant. It is to be understood that, aside or in addition to quadrants other divisions of the contact area are possible. The contact area may be divided into halves, twelfths, along medial/lateral divisions, etc. The data may be provided in real-time and as it changes, the colors in the selected areas could reflect that dynamic.

Data may be presented in other formats, such as bar graphs, line graphs, and the like. The different formats of data presentation may be displayed alone or in combination with other data presentation formats.

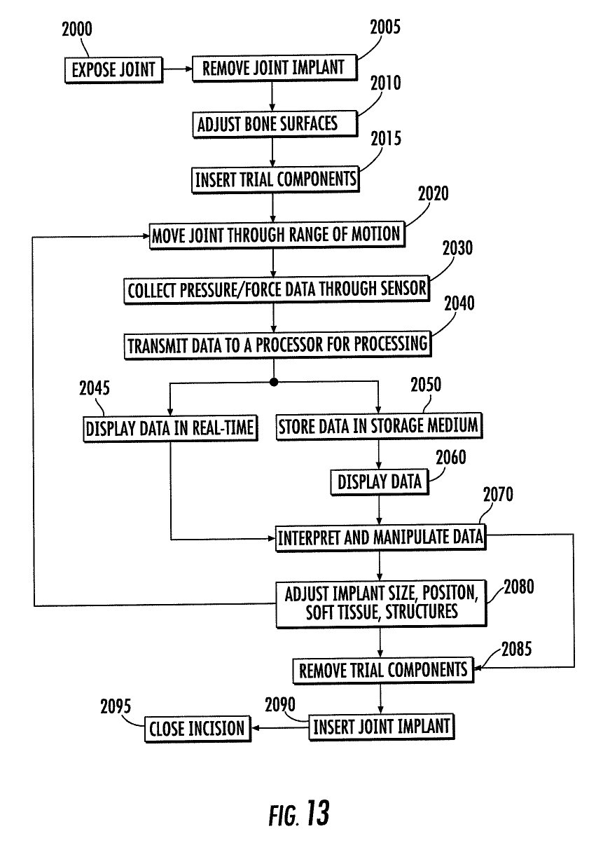

In an embodiment, the method is accomplished as illustrated in FIG. 12. The joint is exposed (box 1000), bone is cut and bone pieces are removed. Spaces left by the removed bone can be replaced through the method. The sensor device(s) and remaining joint replacement devices are then inserted and secured into the joint (box 1015). The joint is then moved through a partial or full range of motion (box 1020). Joint and implant characteristic data is then collected through the conformable sensor (box 1030). The joint data is then transmitted to a processor for processing (box 1040). When transmitted, the data may be stored in storage medium (box 1050), and/or displayed in real-time (box 1045). Stored data may also be retrieved and displayed at a later time (box 1060). After storing and or displaying the data, the data may then be interpreted and/or manipulated (box 1070) using software. The interpretation and manipulation results may also be displayed or otherwise conveyed to the surgeon, who may then adjust the size or position of the implants, bones, or surrounding tissue, based on these collected joint and implant characteristics (box 1080). After adjustment, the surgeon may again test the implant device by moving the joint through a range of motion (box 1020), collect additional data (box 1030), observe the results (box 1070), and make any additional adjustments (box 1080), as discussed above. This procedure of making adjustments and observing the results by collecting data may be repeated as many times as the surgeon deems necessary. Once no further adjustments are needed, the surgeon removes the sensor device(s) (box 1085), inserts the final joint implant in the place of the sensor device (box 1090), and closes the incision (box 1095).

FIG. 13 includes a flow chart illustrating a method of using a sensor device to measure joint characteristics during revision joint replacement surgery in alternative embodiments. The joint is exposed (box 2000), previously implanted replacement devices are removed (box 2005), and bone surface is adjusted (box 2010) by e.g., cutting bone spurs, as needed. The sensor device(s) and the remaining joint replacement implants are inserted and secured into the joint (box 2015). The joint is then moved through a partial or full range of motion (box 2020). Joint and implant characteristic data is then collected through the conformable sensor (box 2030). The joint data is then transmitted to a processor for processing (box 1040). When transmitted, the data may be stored in storage medium (box 2050), and/or displayed in real-time (box 2045). Stored data may also be retrieved and displayed at a later time (box 2060). After storing and or displaying the data, the data may then be interpreted and/or manipulated (box 2070) using software. The interpretation and manipulation results also may be displayed or otherwise conveyed to the surgeon, who may then adjust the size or position of the implants, bones, or surrounding tissue, based on these collected joint and implant characteristics (box 2080). After adjustment, the surgeon may again test the implant device by moving the joint through a range of motion (box 2020), collect additional data (box 2030), observe the results (box 2070), and make any additional adjustments (box 2080), as discussed above. This procedure of making adjustments and observing the results by collecting data may be repeated as many times as the surgeon deems necessary. Once no further adjustments are needed, the surgeon may remove the sensor device(s) (box 2085), re-insert the joint replacement implant (box 2090), and close the incision (box 2095). Alternatively, a new implant may be inserted.

In a further embodiment, more than one sensor device is placed in a joint during joint replacement or implant revision surgery. For example, a sensor device modeled on a patellar implant and another modeled on a femoral implant may be the trial component implanted during a TKA procedure. Joint replacement or revision embodiments including single or multiple sensor devices and joints other than the knee are also contemplated.

In preferred embodiments, operation of the sensor device used during either a joint replacement procedure or a revision joint replacement procedure is as follows. The sensor matrix is arranged directly on the bone surface within a joint. The surface may be either a flat or a curved surface. Direct contact between the sensor and the bone surface provides an accurate reading of the pressures and forces between bone and the implant prosthesis when collecting data.

In a preferred embodiment, the sensor collects pressure and force data from the bone surface within the joint. This data is then converted to output data and transmitted to a storage medium or a display device. The output data may be stored in a storage medium such as a computer, a network, a database, or any other data storage medium known to one of ordinary skill in the art. The output data may also be displayed on a display device in real-time so that a surgeon may observe this real-time output data during surgery and immediately adjust one or more of the implants' size or position, the bone cuts, and the surrounding soft tissue structures (such as when performing retinacular releases) based on the displayed data. The display device may be a computer monitor, a screen, a hand-held display, or other display device known to one of ordinary skill in the art. After making necessary adjustments, additional pressure and force output data may again be collected and sent to the storage and/or display device, and further direct the surgeon to make any additional intraoperative changes.

In still further embodiments, after the data is collected, it is transmitted to a computer navigation system for subsequent storage, display, and/or processing. Computer navigation systems include a combination of cameras, computers, software, and position markers. The position markers are placed on a patient part during surgery. The camera picks up the body part's movement and translates it into angles/position demonstrated on the computer screen. The camera may be a digital camera that produces highly accurate reliable results that are configurable to be used with imaging and processing software and instrumentation. The camera senses the instrumentation's movement, translates the movement into angles and positions, and displays these angles and positions on a display device. The sensor device, when used in conjunction with a computer navigation system, assists and guides a surgeon with making clinical decisions during surgery. The display device may include one or more glare-free monitors that allows for a high degree of flexibility and ease of use in conjunction with the software, camera, and instrumentation. The display device can be attached to an articulating arm or other instrumentation for ease in positioning and articulating the instrumentation through a range of motion. The instrumentation includes controls that easily give a user, particularly a surgeon, complete control of the software, which makes the surgical procedures more efficient. The instrumentation provides a steady flow of information from the sensors to the storage and display device. The instrumentation's functions are also accessible without having to remove the instrumentation from the operating field. Because of the position markers, the navigation system's software can offer the surgeon accurate information in implant alignment, instrument orientation, and soft tissue balancing to facilitate intraoperative choice and reduce outliers. The software can permit the surgeon to maneuver through different screens based on the tracker's position without having to touch a button or screen. The software can also be customizable to adapt to a user's preference or needs. The software can also provide real-time information about implants and surrounding bones and tissue, and can be connected to a storage medium to display previously archived data. The software can be used to create a virtual anatomy by registering a patient's bones' or joint positions at any point in a resurfacing or implant procedure. The digitized bone position information creates a landmark for subsequent work. Reconfigurable kinematic screens provide a surgeon with pre-operative, intra-operative, and post-implant assessments of the patient's joint kinematics in real-time. The software does not isolate a surgeon with one specific surgical path when resurfacing a bone surface, but permits the surgeon to refine a bone surface during the resurfacing phase of an implant procedure. Because the software allows a surgeon to make more accurate bone resections during a knee replacement procedure, the overall operative time is reduced.

In still further embodiments, after the data is collected, stored, and/or displayed, the data may then be interpreted and manipulated using the computer software to display stress distribution and joint contact areas in topographical ‘nomogram’ form. The user may define an area of the conformable sensor as a “mask” and the users definition may be customizable (e.g., a quadrant or quadrants of the conformable sensor). The software may also be used to interpret the pressure and force data and produce other pressure and force data, such as the following:

Mean Pressure (kPa) is the average pressure (within the masks) for each point in time.

Average Mean Pressure (kPa) is the average of the mean pressure across the entire trial (within the masks).

Mean Force (N) is the average force across the entire trail (within the masks).

Peak Pressure (kPa) is the highest pressure value received throughout the trial (within the masks).

Maximum Force (N) is the highest force value received throughout the trial (within the masks).

Maximum Pressure Picture (MPP) is a single picture of the maximum value that each sensor element received throughout the trial. It does not represent any point in time.

Mean Pressure for MPP (kPa) is the average pressure of the MPP (within the masks).

Mean Value Picture (MVP) is a single picture which represents the average of the entire trial across only the loaded sensors. This may give a higher value than mean force, since there may be sensors which are not loaded within the trial.

Force for MVP is the force value of the MVP (within the masks).

Pressure-time integral (kPa*sec) or PTI is the multiple of pressure across the entire time of the trial. It is a single value for the entire trial (within the masks).

Force-time integral (N*sec) or FTI is the multiple of force across the entire time of the trial. This is also referred to as “impulse”. It is a single value for the entire trial (within the masks).

The sensor device in the above embodiments may be wireless, or alternatively attached to a processor or computer by wires. Also, while the discussion above is directed to a TKA procedure, sensor device may alternatively be used in other joint areas or implant components.

In another embodiment, the methods of performing patellofemoral alignment or alignment related to any other joint implant surgery is performed using a sensor device or sensor devices as described above. In this embodiment, however, one or more of the sensor devices also serves as the final implant(s). The final implant sensor device(s) includes or is operatively connected to a power source in situ, is operatively connected to a transceiver and antenna. Through this embodiment, the sensor device, serving as the final implant, communicates data wirelessly to a data processor. The data can be monitored to assess implant alignment continuously or discontinuously thereafter. Using this embodiment, a patient's implant replacement can be monitored while in use and any wear can be detected by a change in the data. Additionally, changes in the patient's health, bone structure, soft tissue health, and the like may be reflected through changes in the data. Under this embodiment of the invention, an implant replacement can be monitored to determine if any non-operative or operative intervention may address changes in the joint or implant structure over time. In a still further embodiment, the sensor device that serves as a final implant also receives wireless communications. Through this embodiment, implementing software within the device can be managed, modified, or replaced. Also, the performance or condition of individual components, including the sensor elements, the sensor matrix, and the conformable sensor may be assessed. In still further embodiments, adjustments to the sensor device and any of these components may be implemented wirelessly.

The following examples are presented to illustrate the practice of specific preferred embodiments.

### EXAMPLES

### Cadaver Protocols

Six fresh cadavers (twelve knees; three males, three females) were obtained for this study. Each knee was tested in its pre-arthroplasty state by applying a conformable mat sensor and evaluating pressures while taking the knee through an arc of motion (0-120 degrees).

Data Collection: Simultaneous real-time force, pressure and video data was collected for each knee, extracted from specified regions of the patella, and graphically represented versus time over the range of dynamic flexion. Analysis was performed on data for static instances in 0, 30, 60, 90, and 120 degrees of flexion. In the case of the instrumented patellar trial, values were recorded from each of four quadrants of the component for a given flexion angle. In the case of the conforming mat, pressure readings were recorded for medial and lateral femoral condyles. Statistical analysis was directed towards detection of differences in pressure distribution effected by TKR in comparison to the natural knee, as well as differences effected by femoral rotation, patellar thickness, lateral release, and joint line position in comparison to the baseline TKR data.

While collecting pressure distribution data, simultaneous video documented the flexion angle accompanying each measurement. After completing the measurements for the natural knee, the sensor was removed and the patellar cut for resurfacing was made. At this point the experiment was repeated using the instrumented patellar trial.

After the measurements were completed, bone cuts were made for a total knee replacement utilizing standard jigs and implants. The 12 knees were stratified randomly into three groups, each containing a male and a female, with different femoral rotation (internal, neutral, external). Following replacement, the same protocol was followed measuring pressure distributions throughout a 0-120 degree arc of motion with the following independent variables: a) three different sizes of patellar trials in progression to determine the effect of patellar thickness on pressure distribution, b) standardized lateral release extending from the vastus lateralis to the joint line, passing 1-2 cm lateral to the patella, c) the effect of implanting a gender-specific femoral component on patellofemoral pressures for each of the independent variables, and d) the differences between measurements made using the conformable mat sensor and the instrumented patellar trial.

The femur was then re-cut and the tibial spacer augmented to elevate the joint line by 5 millimeters, and the same protocol was followed, including variation of patellar thickness, and testing the instrumented patellar trial.

All references cited herein are incorporated by reference in their entirety as if fully set forth herein.

It is understood, therefore, that the invention is not limited to the particular embodiments disclosed, but is intended to cover all modifications which are within the spirit and scope of the invention as defined by the appended claims; the above description; and/or shown in the attached drawings.

What is claimed is:

1. A sensor device comprising:
a base plate having
a base plate bottom surface adapted to contact a joint surface, and
a base plate top surface;
a conformable sensor including
a sensor matrix that includes individual sensor elements, and
a conformable mat,
wherein the conformable mat supports the sensor matrix;
an implant having an implant bottom surface and an implant top surface,
the conformable sensor operably connected to the implant and being associated with either the base plate top surface, the implant bottom surface, the implant top surface, or the base plate top surface and the implant bottom surface,
the base plate top surface facing and operatively connected to the implant bottom surface.

2. The sensor device of claim 1, further comprising a data transmission device, wherein the sensor matrix collects joint data and the data transmission device transmits the joint data from the sensor matrix to a data processor.

3. The sensor device of claim 2, the joint data comprises data selected from the group consisting of stress, pressure, and force data.

4. The sensor device of claim 1, wherein at least a portion of the conformable sensor is embedded in the implant.

5. The sensor device of claim 1, wherein the conformable sensor is integral with the implant.

6. The sensor device of claim 1, wherein at least a portion of the sensor matrix is embedded in the conformable mat.

7. The sensor device of claim 1, the implant top surface having a dome-shape.

8. The sensor device of claim 1, wherein the individual sensor elements include a force sensor selected from the group consisting of a pressure sensor, a pressure transducer, a capacitive transducer, a capacitive sensor, a resistive sensor, a piezoelectric sensor, a force transducer, a strain gauge, and a microelectromechanical contact stress sensor.

9. The sensor device of claim 1, wherein the force sensor is a capacitive sensor.

10. The sensor device of claim 1, wherein the force sensor is a piezoelectric sensor.

11. The sensor device of claim 1, wherein the force sensor is a force transducer.

12. The sensor device of claim 1, wherein the sensor device is shaped like a patellar implant.

13. A sensor device comprising:
a base plate having
a base plate bottom surface adapted to contact a joint surface, and
a base plate top surface;
a conformable including a sensor matrix that includes individual sensor elements arranged in a conformable mat,
an implant having an implant bottom surface and an implant top surface,
the conformable sensor being operably connected to the implant and associated with either the base plate top surface, the implant bottom surface, the implant top surface, or the base plate top surface and the implant bottom surface,
the base plate top surface facing and operatively connected to the implant bottom surface.

14. The sensor device of claim 13, further comprising a data transmission device, wherein the sensor matrix collects joint data and the data transmission device transmits the joint data from the sensor matrix to a data processor.

15. The sensor device of claim 14, the joint data comprising data selected from the group consisting of stress, pressure, and force data.

16. The sensor device of claim 13, wherein at least a portion the conformable sensor is embedded within the implant.

17. The sensor device of claim 13, the implant top surface having a dome-shape.

18. The sensor device of claim 13, wherein the individual sensor elements include a force sensor selected from the group consisting of a pressure sensor, a pressure transducer, a capacitive transducer, a capacitive sensor, a resistive sensor, a piezoelectric sensor, a force transducer, a strain gauge, and a microelectromechanical contact stress sensor.

19. The sensor device of claim 13, wherein the force sensor is a capacitive sensor.

20. The sensor device of claim 13, wherein the force sensor is a piezoelectric sensor.

21. The sensor device of claim 13, wherein the force sensor is a force transducer.

22. The sensor device of claim 13, wherein the sensor device is shaped like a patellar implant.

23. A method of using a sensor device to measure joint characteristics during joint replacement or joint implant revision, the method comprising:
a) providing sensor device including
a base plate having a base plate bottom surface adapted to contact a joint surface, and a base plate top surface;
a conformable sensor including a sensor matrix that includes individual sensor elements; and a conformable mat, the conformable mat supports the sensor matrix;
an implant having an implant bottom surface and an implant top surface, the conformable sensor being operably connected to the sensor implant and associated with either the base plate top surface, the implant bottom surface, the implant top surface, or the base plate top surface and the implant bottom surface, the base plate top surface facing and operatively connected to the implant bottom surface,
b) making an incision in a patient to expose the joint and removing one of the group consisting of bone and pre-existing implants,
c) inserting the sensor device and remaining joint implants required for the joint replacement or joint implant revision into the joint,
d) moving the joint through a partial or full range of motion,
e) collecting joint data through the sensor matrix,
f) making necessary adjustments based on the joint data,
g) repeating steps d)-f) until the joint, the sensor implant, and the remaining joint implants are in a desirable position,
h) removing the sensor device,
i) inserting a final implant in place of the sensor device, and
j) closing the incision.

24. The method of claim 23, further comprising, between b) and c), adjusting bone surface as needed.

25. The method of claim 24, wherein adjusting the bone surface further comprises cutting bone spurs from bones on the joint surface.

26. The method of claim 23, wherein f) further comprises at least one of adjusting the joint, soft tissue, the remaining joint implants, and the sensor device based on the joint data.

27. The method of claim 23, wherein the sensor device is shaped like a patella and the final implant is a patellar implant.

28. A sensor device comprising:
a base plate having
a base plate bottom surface adapted to contact a joint surface, and
a base plate top surface;
a conformable sensor including
a sensor matrix that includes individual sensor elements, and
a conformable mat,
wherein the conformable mat supports the sensor matrix;
an implant having an implant bottom surface and an implant top surface,
the conformable sensor operably connected to the implant and being associated with either the base plate top surface, the implant bottom surface, the implant top surface, or the base plate top surface and the implant bottom surface,
the base plate top surface facing and operatively connected to the implant bottom surface;
the sensor device further comprising a transceiver and antenna to wirelessly transmit data to a data processor or wirelessly receive communications.

29. A joint replacement implant collection comprising:
i) a sensor device shaped in the form of a final implant that will be associated with one bone within a joint, the sensor device including
a base plate having
a base plate bottom surface adapted to contact a joint surface, and
a base plate top surface;
a conformable sensor including
a sensor matrix that includes individual sensor elements, and
a conformable mat,
wherein the conformable mat supports the sensor matrix;
an implant having an implant bottom surface and an implant top surface,
the conformable sensor operably connected to the implant and being associated with either the base plate top surface, the implant bottom surface, the implant top surface, or the base plate top surface and the implant bottom surface,
the base plate top surface facing and operatively connected to the implant bottom surface; and
ii) remaining implants in the form of implants associated with the remaining bones in the joint.

30. The joint replacement implant collection of claim 29, wherein the sensor device is shaped like a patellar implant.

31. The joint replacement implant collection of claim 29, wherein the sensor device is also the final implant.

32. The joint replacement implant collection of claim 31, wherein the sensor device further comprises a transceiver and antenna for wireless communications.

33. The joint replacement implant collection of claim 29, wherein the sensor device further comprises a transceiver and antenna for wireless communications.

34. A joint replacement implant collection comprising:
i) a sensor device shaped in the form of a final implant that will be associated with one bone within a joint, the sensor device including
a base plate having
a base plate bottom surface adapted to contact a joint surface, and
a base plate top surface;
a conformable sensor including
a sensor matrix that includes individual sensor elements arranged in a conformable mat;
an implant having an implant bottom surface and an implant top surface,
the conformable sensor operably connected to the implant and being associated with either the base plate top surface, the implant bottom surface, the implant top surface, or the base plate top surface and the implant bottom surface,
the base plate top surface facing and operatively connected to the implant bottom surface; and
ii) remaining implants in the form of implants associated with the remaining bones in the joint.

35. The joint replacement implant collection of claim 34, wherein the sensor device is shaped like a patellar implant.

36. The joint replacement implant collection of claim 34, wherein the sensor device is also the final implant.

37. The joint replacement implant collection of claim 36, wherein the sensor device further comprises a transceiver and antenna for wireless communications.

38. The joint replacement implant collection of claim 34, wherein the sensor device further comprises a transceiver and antenna for wireless communications.
